(19) United States
(12) Patent Application Publication
(10) Pub. No.: US 2016/0016984 A1
(43) Pub. Date: Jan. 21, 2016

(54) POSITIVE AND NEGATIVE EXTRACTION DEVICE AND EXTRACTION METHOD

(71) Applicant: AMOREPACIFIC CORPORATION, Seoul (KR)

(72) Inventors: Jun Seong Park, Gyeonggi-do (KR); Dong Hyun Kim, Gyeonggi-do (KR); Eun Jeong Moon, Seoul (KR); Sun Hye Yu, Gyeonggi-do (KR); Eun Joo Kim, Gyeonggi-do (KR); Duck Hee Kim, Seoul (KR); Han Kon Kim, Gyeonggi-do (KR); Myung Kyoo Kim, Gyeonggi-do (KR)

(21) Appl. No.: 14/807,971

(22) Filed: Jul. 24, 2015

Related U.S. Application Data
(62) Division of application No. 13/379,890, filed on Dec. 21, 2011, now abandoned.

(51) Int. Cl. C07H 7/06; C07H 1/08; B01D 11/02

(52) U.S. Cl. CPC C07H 7/06; B01D 11/0215; C07H 1/08

(57) ABSTRACT

Provided is a pressure-cycling type of extraction device comprising: an extraction unit for accommodating and extracting an extraction target substance; a compression unit for increasing the pressure inside the extraction unit; and a decompression unit for decreasing the pressure inside the extraction unit; wherein the pressure inside the extraction unit is increased and decreased by alternately operating the compression unit and the decompression unit, and the extraction unit comprises a bubbling nozzle for supplying bubbles into the extraction target substance. Further provided is a pressure-cycling type of extraction method in which a compression process and a decompression process are carried out alternately, and compositions produced by the method. The extraction device and method allow low temperature extraction, permit outstanding extraction efficiency and can prevent colour changes, olfactory changes and thermal denaturation of the extraction target substance.

### CROSS-REFERENCE TO RELATED APPLICATIONS

This application is a divisional application of U.S. patent application Ser. No. 13/379,890, filed Dec. 21, 2011, and also claims priority to Korean Patent Application 10-2009-0057901, filed Jun. 26, 2009, both of which are hereby incorporated by reference in their entirety.

### TECHNICAL FIELD

The present disclosure relates to a pressure-cycling type of extraction device and a pressure-cycling type of extraction method.

### BACKGROUND ART

In general, hot water extraction of extracting using hot water is commonly employed for extraction of natural products or medicinal herbs. However, the hot water extraction process is problematic in that the extraction target substance may be degraded by heat, resulting in discoloration or bad smell or decomposition of the active ingredient.

To solve the thermal degradation problem, low-temperature extraction is often used. Although this method can prevent the degraded of the active ingredient, it is commercially inapplicable since the extraction efficiency is decreased greatly. And, an extraction method using an organic solvent is problematic in that the solvent remaining after the extraction may be harmful to the human body.

### DISCLOSURE

### Technical Problem

The present disclosure is directed to providing a pressure-cycling type of extraction device.

The present disclosure is also directed to providing a pressure-cycling type of extraction method.

The present disclosure is also directed to providing an extract extracted by a pressure-cycling type of extraction device and a composition containing the same.

### Technical Solution

In one general aspect, the present disclosure provides a pressure-cycling type of extraction device comprising: an extraction unit for accommodating and extracting an extraction target substance; a compression unit for increasing the pressure inside the extraction unit; and a decompression unit for decreasing the pressure inside the extraction unit; wherein the pressure inside the extraction unit is increased and decreased by alternately operating the compression unit and the decompression unit, and the extraction unit comprises a bubbling nozzle for supplying bubbles into the extraction target substance.

In another general aspect, the present disclosure provides a pressure-cycling type of extraction method, comprising carrying out a compression process and a decompression process alternately.

### Advantageous Effects

The extraction device and method using pressure cycling according to the present disclosure are advantageous in that extraction is possible at relatively low temperature and extraction efficiency is very high. Also, bad smell or thermal degradation of the extraction target substance can be prevented. The extraction device and method can be widely applied in the fields of foods and cosmetics.

### DESCRIPTION OF DRAWINGS

FIG. 1 schematically shows a cross-section of an extraction device according to an exemplary embodiment of the present disclosure;

FIG. 2 is an enlarged view showing engagement of an extraction bath and an upper plate in a pressure-cycling type of extraction device;

FIG. 3 shows extraction ratio of different extraction methods with temperature; and

FIG. 4 shows extraction ratio of different extraction methods with extraction time.

Detailed Description of Main Elements

| | |
|---|---|
| 100: extraction unit | 110: extraction bath |
| 111: bubbling nozzle | 120: upper plate |
| 121: spray nozzle | 200: compression unit |
| 300: decompression unit | 400: temperature controller |
| 500: circulation pump | |

### BEST MODE

A pressure-cycling type of extraction device according to an embodiment of the present disclosure comprises: an extraction unit for accommodating and extracting an extraction target substance; a compression unit for increasing the pressure inside the extraction unit; and a decompression unit for decreasing the pressure inside the extraction unit. The compression unit and the decompression unit operate alternatingly to increase or decrease the pressure inside the extraction unit. The pressure-cycling type of extraction device according to the present disclosure may adjust the number and frequency of compression and decompression according to the properties of the extraction target substance. Also, the extraction condition may be optimized by adjusting compression and decompression time according to the properties of the extraction target substance.

In an exemplary embodiment, the extraction unit may comprise a bubbling nozzle and/or a spray nozzle to improve extraction efficiency.

The bubbling nozzle facilitates stirring of the extraction target substance by means of bubbles formed by the nozzle. Inside the extraction device, there may occur local differences of extraction condition such as temperature, pressure, etc. By supplying bubbles of air or inert gas by means of the bubbling nozzle, the extraction target substance may be stirred more effectively and a more uniform extract may be obtained. Also, the bubbling nozzle allows extraction at low temperature by improving the extraction efficiency. The extraction at low temperature allows the volatile components of the extraction target substance to remain and, thus, the quality of the extract can be significantly improved. In an exemplary embodiment, the inert gas supplied by the bubbling nozzle is nitrogen or helium, specifically nitrogen.

The spray nozzle sprays liquid to the extraction target substance while the extraction is carried out, thus removing foams generated during the extraction. In an exemplary embodiment, the spray nozzle sprays an extraction solvent or water, more specifically low-temperature water. In an exemplary embodiment, the extraction solvent sprayed by the spray nozzle is C₁-C₅ lower alcohol. The spray nozzle makes it unnecessary to use a defoaming agent.

In an exemplary embodiment, the extraction unit may be in the form of, for example an integral tank, although not limited thereto. When the extraction unit is in the form of an integral tank, it may have inlet ports for supplying the extraction target substance, the solvent, etc. and an outlet port for discharging the extract to outside.

In another exemplary embodiment, the extraction unit may comprise an extraction bath wherein the extraction target substance is held and an upper plate. The extraction bath holds the extraction target substance and a solvent, if necessary. The upper plate is disposed on the extraction bath and closes the extraction bath. The extraction bath may be engaged with the upper plate by any means that allow the extraction target substance held in the extraction bath to be isolated, without special limitation. For example, the extraction bath and the upper plate may be engaged with each other by protrusions formed on the top surface of the extraction bath and protrusions formed on the bottom surface of the upper plate.

The extraction device may further comprise a temperature controller. The temperature controller heats the extraction target substance held in the extraction unit. By heating the extraction target substance using the temperature controller, extraction efficiency may be improved.

In an exemplary embodiment, the extraction device alternately provides pressurizing and depressurizing conditions. A compression unit may increase pressure by injecting gas into the extraction unit. The gas injected by the compression unit may be air or inert gas, specifically nitrogen (N₂) or helium (He) gas, more specifically nitrogen gas, although not being limited thereto. The decompression unit may decrease pressure by applying vacuum to the extraction unit or discharging air through a vent.

The present disclosure also provides a pressure-cycling type of extraction method, comprising carrying out a compression and a decompression alternately.

In an exemplary embodiment, the pressure-cycling type of extraction method may comprise: (a) a compression process increasing pressure; and (b) a decompression process decreasing pressure, alternatingly. The extracting method may further comprise: (c) supplying bubbles to the extraction target substance during said compression and/or decompression.

In another exemplary embodiment, the extraction method may further comprise: (d) spraying liquid to the extraction target substance during the extraction.

The extracting method may be a solvent extraction method of extracting the extraction target substance by immersing it in a solvent. The solvent used in the extracting method may be water or C₁-C₅ lower alcohol, more specifically water, although not being limited thereto.

The pressure during said compression may be, for example, 1-10 kgf/cm², specifically 1.5-3 kgf/cm², although not being limited thereto. Depending on situations, an intermediate pressure of about 5 kgf/cm² may be used. The pressure during said decompression may be 100-760 mmHg, specifically 500-700 mmHg. Depending on situations, the depressurizing may be performed in vacuum. In the extraction method according to the present disclosure, the pressurizing and depressurizing may be performed alternatingly depending on the properties of the extraction target substance. If necessary, the conditions of the compression and decompression processes may be set differently.

The number and frequency of the compression and decompression processes are not specially limited and may be set differently according to the properties of the extraction target substance. In an exemplary embodiment, each of the compression and decompression processes may be performed for 10 minutes to 1 hour, specifically for 30 minutes. In another exemplary embodiment, the compression and decompression processes may be repeated 1-30 times, specifically 2-10 times, more specifically 4-5 times. By repeating the compression and decompression processes 2-3 times, sufficient extraction efficiency may be achieved.

Since compression and decompression are performed alternatingly, the extraction method according to the present disclosure allows extraction at a relatively lower temperature than the hot water extraction method. Accordingly, the extraction temperature may be 0-100° C., specifically 30-85° C. The extracting method allows effective extraction even at a relatively low temperature of 50-75° C. Depending on situations, cold extraction may also be carried out.

The present disclosure further provides an extract extracted using the extraction device or by the extraction method according to the present disclosure. Since the extract according to the present disclosure is nearly free from discoloration, bad smell or thermal degradation, the inherent fragrance and nutritional ingredients of the extraction target substance may be maintained effectively. The extract may be used as an active ingredient or an additive in foods, cosmetics or pharmaceuticals. In an exemplary embodiment, the present disclosure provides a food composition comprising the extract. In another exemplary embodiment, the present disclosure provides a cosmetic composition comprising the extract.

Now, extraction devices using pressure cycling according to exemplary embodiments of the present disclosure will be described in more detail with reference to the attached drawings.

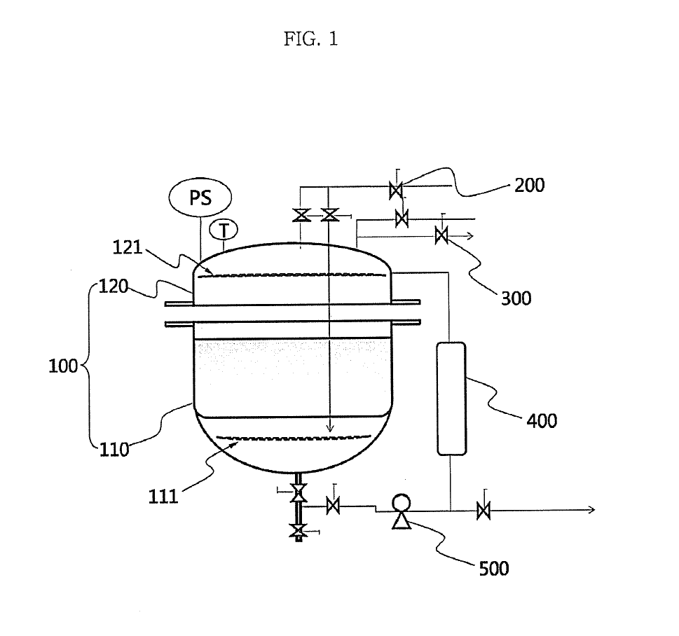

FIG. 1 schematically shows a cross-section of an extraction device according to an exemplary embodiment of the present disclosure. The extraction device comprises an extraction unit 100 wherein an extraction target substance is extracted, a compression unit 200 increasing the pressure inside the extraction device, and a decompression unit 300 decreasing the pressure inside the extraction device.

The extraction unit 100 comprises an extraction bath 110 accomodating the extraction target substance, and an upper plate 120 serving as a lid of the extraction bath 110. A bubbling nozzle 111 is provided at the lower portion of the extraction bath 110. The bubbling nozzle 111 supplies air bubbles during the extraction process to facilitate stirring of the extraction target substance. The upper plate 120 has a spray nozzle 121 at the lower portion thereof. By spraying low-temperature water, the spray nozzle 121 removes foams generated during the extraction.

The compression unit 200 and the decompression unit 300 are connected to the upper plate 120. The compression unit 200 increases the pressure inside the extraction device by supplying air or nitrogen (N₂) gas. The decompression unit 300 decreases the pressure inside the extraction device by applying vacuum or discharging air through a vent. The operation time and frequency of the compression unit 200 and the decompression unit 300 are controlled by a pressure controller (not shown). A temperature controller 400 is connected to the extraction device. An extract heated by the temperature controller 400 is circulated into the extraction bath 110 by a circulation pump 500.

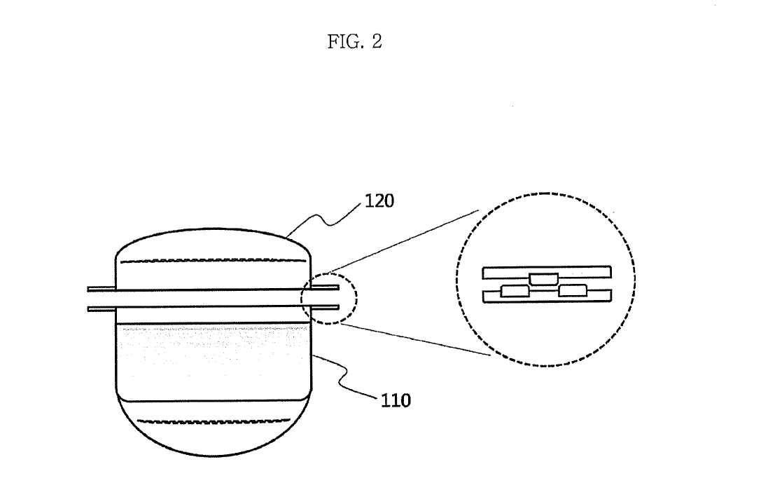

FIG. 2 shows engagement of the extraction bath and the upper plate in a pressure-cycling type of extraction device according to an embodiment of the present disclosure. The extraction bath 110 has two protrusions formed on its top surface, which are engaged with a protrusion formed on the bottom surface of the upper plate 120.

### MODE FOR INVENTION

The examples and experiments will now be described. The following examples and experiments are for illustrative purposes only and not intended to limit the scope of the present disclosure.

### Example 1

### Pressure Cycling Extraction

Puerarin was extracted from the root of kudzu using the extraction device shown in FIG. 1. Specifically, the root of kudzu was immersed in 10 times the volume water and extracted for 2 hours while alternatingly pressurizing and depressurizing with 30-minute intervals. The pressure during compression was 2 kgf/cm² and the pressure during decompression was 600±50 mmHg, and the extraction temperature was set at 75° C.

### Comparative Example 1

### Hot Water Extraction

Puerarin was extracted from the root of kudzu by hot water extraction. Specifically, the root of kudzu was immersed in 10 times the volume water and extracted for 2 hours at 75° C. Compression or decompression was not carried out during the extraction.

### Test Example 1

### Comparison of Extraction Ratio at Different Temperatures

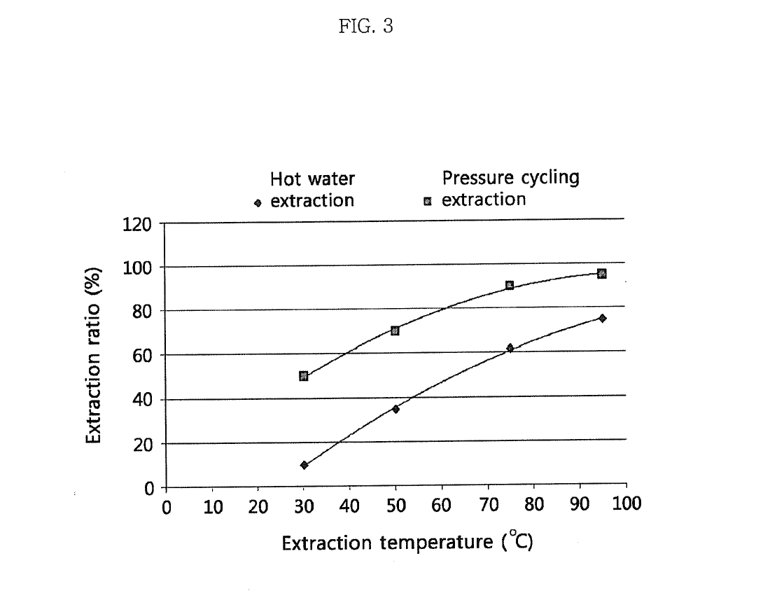

Puerarin was extracted from the root of kudzu in the same manner as described in Example 1 and Comparative Example 1 and extraction ratio was measured. But, the extraction time was 4 hours, compression and decompression were repeated with 1-hour intervals, and the extraction temperature was varied from 30 to 100° C. The result is shown in FIG. 3. In FIG. 3, the extraction ratio (%) is given relative to the methanol reflux extraction according to the Korean Pharmacopoeia.

Referring to FIG. 3, the pressure cycling extraction exhibits 20-40% higher extraction ratio than the hot water extraction. Especially, the extraction ratio is good at the low temperature range. For example, the extraction ratio is less than 10% at 30° C. and less than 40% at 50° C. for the hot water extraction. In contrast, the pressure cycling extraction according to the present disclosure exhibits an extraction ratio of 50% or higher at 30° C. and an extraction ratio reaching 70% at 50° C.

It can be seen that the pressure cycling extraction according to the present disclosure allows effective extraction at relatively low temperatures where extraction is hardly accomplished by the existing hot water extraction method. Accordingly, the pressure cycling extraction according to the present disclosure can be effectively utilized for extraction of thermally susceptible ingredients.

### Test Example 2

### Change of Extract with Time

Change of the extracts extracted by hot water extraction and pressure cycling extraction was observed in order to investigate the efficiency of pressure cycling extraction.

Hot water extraction and pressure cycling extraction were performed in the same manner as described in Test Example 1. In each case, extraction time was 2 hours. The obtained extracts were filtered through a 0.45-μm filter to remove microorganisms and precipitation was observed in a refrigerator. The result is shown in Table 1.

TABLE 1

| | Days | | | | | | | |
|---|---|---|---|---|---|---|---|---|
| Extraction | 1 day | | 7 days | | 15 days | | 30 days | |
| temperature | HWE | PCE | HWE | PCE | HWE | PCE | HWE | PCE |
| 30° C. | — | — | — | — | — | — | — | — |
| 50° C. | — | — | — | — | * | — | * | — |
| 75° C. | — | — | — | — | * | * | * | * |
| 95° C. | * | — | ** | ** | *** | *** | *** | *** |

HWE: hot water extraction
PCE: pressure cycling extraction
* slightly hazy,
** precipitation observed,
*** slight precipitation,
**** considerable precipitation,
***** severe precipitation

As seen from Table 1, significant differences in precipitation were observed depending on the extraction temperature. The temporal stability was relatively superior for pressure cycling extraction when the extraction temperature was relatively low at 50-75° C.

### Test Example 3

Comparison of extraction ratio for hot water extraction, extraction by pressurizing only, extraction by depressurizing only and pressure cycling extraction

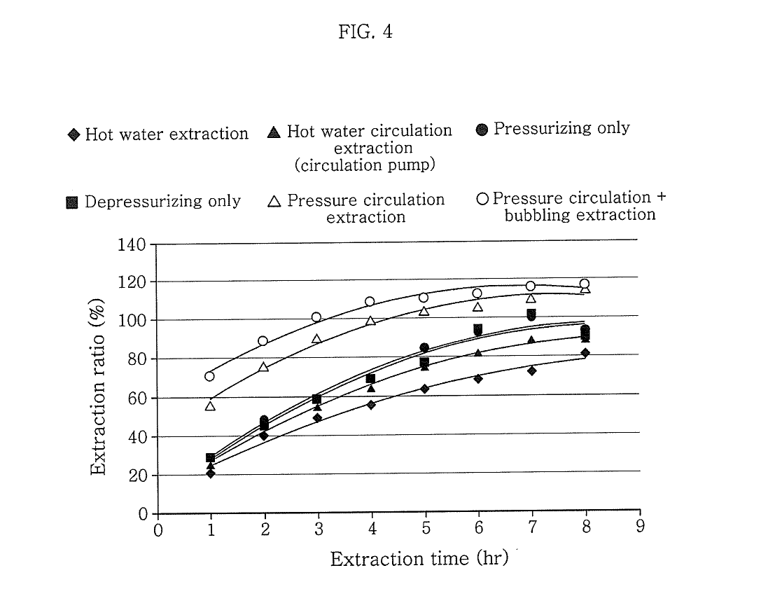

Extraction ratio was compared for hot water extraction, extraction by pressurizing only, extraction by depressurizing only and pressure cycling extraction. Extraction time was 8 hours and extraction ratio was measured every hour by taking samples. The extraction temperature was set at 75° C. since some ingredients may not be extracted at lower temperatures and thermally weak ingredients may be degraded at higher temperatures. The specific extraction condition is shown in Table 2, and the result is shown in FIG. 4.

TABLE 2

| | Extraction | Pressurizing | Depressurizing | |
|---|---|---|---|---|
| Extraction method | temperature | condition | condition | Note |
| Hot water | 75° C. | — | — | |
| extraction | | | | |
| Hot water | 75° C. | — | — | Hot water extraction |
| circulation | | | | with circulation |
| extraction | | | | pump |
| Pressurizing only | 75° C. | 2 kgf/cm² | — | |
| Depressurizing | 75° C. | — | 600 ± 50 mmHg | |
| only | | | | |
| Pressure cycling | 75° C. | 2 kgf/cm² | 600 ± 50 mmHg | Alternatingly |
| extraction | | | | pressurizing and |
| | | | | depressurizing with |
| | | | | 30-minute intervals |
| Pressure cycling + | 75° C. | 2 kgf/cm² | 60 ± 50 mmHg | Releasing vacuum |
| bubbling extraction | | | | Injecting nitrogen by |
| | | | | bubbling |
| | | | | Alternatingly |
| | | | | pressurizing and |
| | | | | depressurizing with |
| | | | | 30-minute intervals |

Referring to FIG. 4, hot water circulation extraction wherein a circulation pump was used for stirring was exhibited better extraction efficiency than hot water extraction, close to those of pressurizing only and depressurizing only. The extraction yield attained in 8 hours with hot water extraction was achieved only in 2 hours with pressure cycling extraction. When bubbling was combined with pressure cycling extraction, the maximum extraction yield was attained even faster than simple pressure cycling extraction. The pressure cycling extraction combined with bubbling was effective in extracting thermally weak ingredients at low temperature, with remarkably higher efficiency than other methods.

### INDUSTRIAL APPLICABILITY

The extraction device and method according to the present disclosure allow effective extraction of natural products, medicinal herbs, etc. without discoloration, bad smell or thermal degradation of the extraction target substance.

1-6. (canceled)

7. A pressure-cycling type of extraction method for extracting an extraction target substance by immersing in a solvent,
comprising carrying out a compression process and a decompression process alternately,
wherein bubbles are supplied to the extraction target substance during the extraction,
wherein the compression process increases to 1.5˜3 kgf/cm² and the decompression process decreases pressure to 100-760 mmHg.

8. The pressure-cycling type of extraction method according to claim 7, further comprising spraying liquid to the extraction target substance during the extraction.

9. The pressure-cycling type of extraction method according to claim 7, wherein the solvent is water or C₁-C₅ lower alcohol.

10. (canceled)

11. The pressure-cycling type of extraction method according to claim 7, wherein the compression process and the decompression process are performed for 10 minutes to 1 hour, individually, and repeated 2-10 times.

12. The pressure-cycling type of extraction method according to claim 7, wherein extraction temperature is 30-85° C.

13-15. (canceled)
